(19) United States
(12) Patent Application Publication
(10) Pub. No.: US 2017/0067884 A1
(43) Pub. Date: Mar. 9, 2017

(54) Method for the Early Detection of Autism Spectrum Disorder by use of Metabolic Biomarkers

(71) Applicant: Greenwood Genetic Center, Greenwood, SC (US)

(72) Inventors: Anand K. Srivastava, Greenwood, SC (US); Roger E. Stevenson, Greenwood, SC (US)

(21) Appl. No.: 15/251,541

(22) Filed: Aug. 30, 2016

Related U.S. Application Data
(60) Provisional application No. 62/213,871, filed on Sep. 3, 2015.

(51) Int. Cl. G01N 33/53; G01N 33/487

(52) U.S. Cl. CPC G01N 33/5308; G01N 33/487; G01N 2800/28

(57) ABSTRACT

Methods for the identification of autism spectrum disorder in children about 10 years or less of age are described. Methods include testing a sample from a subject for the level of specific biomarker metabolites that have been shown to be different in children with autism spectrum disorder and children that are developing typically. The methods can be utilized as a quick and reliable screening tool for ASD, a diagnostic test for ASD, a measure to monitor treatment of ASD, and may provide a unifying model for the genetic heterogeneity of ASD.

### CROSS REFERENCE TO RELATED APPLICATION

This application claims filing benefit of U.S. Provisional Patent Application Ser. No. 62/213,871 having a filing date of Sep. 3, 2015, which is incorporated herein by reference in its entirety.

### BACKGROUND

Autism Spectrum Disorder (ASD, also collectively referred to herein as autism) constitutes an aberration in function of the central nervous system with numerous dimensions. Subjects are often frustrated in communication and challenged in forming relationships. Autism is a common public health phenomenon that is distributed throughout all strata of the population, with costly and lifelong impact due to limitations on productivity, vulnerability to discrimination and exploitation, and some measure of dependency requiring supervision, support and protection.

Disturbances in three categories of behavior (reciprocal social interactions, verbal and nonverbal communications, and age appropriate activities and interests) are considered hallmarks of ASD. Standardized criteria for autism as defined in the American Psychiatric Association's Diagnostic and Statistical Manual, IVth Edition (DSM IV-TR) and autism spectrum disorder as defined in the Diagnostic and Statistical Manual, Vth Edition (DSM-5) may be assessed based on parental or caregiver interview and/or examiner observations using the Autism Diagnostic Interview, Revised (ADI-R), the Autism Diagnostic Observation Schedule (ADOS), and/or other Autism Diagnostic Instruments. Such testing is typically conducted at about age 3 years.

The number of children diagnosed with ASD has greatly increased in recent decades. At the midpoint of the 20th Century, autism was narrowly defined and uncommonly diagnosed (with a prevalence of about four per 10,000). Greater awareness, availability of services, changes in diagnostic criteria to include a broader spectrum of neurodevelopmental abnormalities, and possibly other factors have contributed to the greater than 30-fold increase in the frequency with which ASD is being currently diagnosed. The prevalence of ASD is currently considered to be above 1% in the U.S. population under 8 years of age (Centers for Disease Control and Prevention, 2009, 2014). An additional extraordinary aspect of the epidemiology is the three-fold to six-fold excess of males.

Unfortunately, the underlying causes of ASD remain elusive, and current diagnostic protocol is limited to behavioral examination as no laboratory finding has been consistently abnormal in ASD. Several biochemical markers (e.g., hypersertoninemia, urinary catabolites, and oxidative metabolism markers) have been inconsistently associated with autistic traits, but a well-defined biomarker screening protocol for ASD susceptibility or presence has not been obtained. For instance, plasma serotonin levels may be elevated in affected individuals and first-degree relatives. In addition, elevated lactate in the brains of individuals with ASD has been demonstrated by magnetic resonance spectroscopic imaging and it has been proposed that metabolic vulnerability to oxidative stress may be an autism susceptibility factor. It has also been suggested that the skewed male: female ratio in autism may be explained by sex-specific responses to the neuropeptides, oxytocin and vasopressin. Impaired utilization of tryptophan as an energy source by lymphoblasts from children with autism has also provided a clue to a metabolic anarchy that lurks within the biochemistry of autism. While these isolated findings have provided additional insight into the biochemical pathways that may be involved in ASD, a reliable testing protocol with demonstrated broad-based success in ASD diagnosis has not been described.

There remains a need for a laboratory test that can offer a reliable confirmation of the clinical diagnosis of ASD and/or to provide a route for an efficient screening of individuals with behavioral features of ASD, and permit the earlier diagnosis of ASD. Because of the absence of consistent physical findings in autism and the uncertainty of the diagnosis in the first years of life, a laboratory test that improves diagnosis of autism, particularly at an early age, would be of great benefit.

### SUMMARY

According to one embodiment, disclosed is a method for early detection of ASD in a subject. For instance, a method can include obtaining a sample, e.g., a blood plasma sample, from a subject, and more specifically from a subject that is about 10 years of age or less. The method also includes determining the concentration level of a plurality of biomarker metabolites in the sample, with the plurality of biomarker metabolites including one of 12-HETE and 15-HETE and also including one of sphingosine and choline. Upon the determination that the concentration level of each of the biomarker metabolites is significantly above or below that of a predetermined control level or control range, the subject can be identified as having or is at risk for developing autism and can be monitored for or treated for autism spectrum disorder. For instance, in one embodiment, the concentration level of the biomarker metabolite can differ from a control level by about 30% or more to signify a significant difference between the concentration level in the test sample and that of the normal control. According to another embodiment ratios of two metabolites can be utilized to determine the presence of ASD. For instance, a ratio of two metabolites in a test sample can be compared to a control ratio of the same two metabolites. A finding that the ratios differ by about 30% or more can signify that the subject is affected with ASD.

According to another embodiment, a testing protocol can include determining the global plasma metabolome of a subject. An analysis of the global plasma metabolome showing a significant difference as compared to a control metabolome as determined by principal component analysis or other comparable statistical analytical methods can signify that the subject is at risk or is affected with ASD and the subject can be monitored or treated for ASD.

The methods described may be utilized as a screening procedure, as a diagnostic test, and/or as a measure to monitor treatment. In one embodiment, following the diagnosis, treatment can include modification of the metabolite level in the subject through, e.g., supplementation in those cases in which the metabolite level is below the control level for that metabolite or alternatively by decreasing the targeted metabolite level in those cases in which the metabolite level is above the control level for that metabolite. By way of example, a metabolite level can be decreased by administration to the subject of an antibody, an inhibitor or an antagonist specific for that metabolite, by dietary modification, or the like.

### BRIEF DESCRIPTION OF THE FIGURES

A full and enabling description of the present disclosure, including the best mode thereof to one skilled in the art, is set forth more particularly in the remainder of the specification, which includes reference to the accompanying figures, in which:

FIG. 1 is a heat map showing 38 metabolites that are increased and 10 metabolites that are decreased in the plasma of 50 children with ASD ages 2-10 years (ASD patients) in comparison to levels of these metabolites in 16 age-matched children who are developing typically (Controls). The metabolite level (median scaled only without log transformation) is scaled accordingly. Black and grey indicate high and low levels, respectively. Data are derived from global metabolome analysis.

FIG. 2 illustrates jitter plot of levels of 15 representative metabolites in plasma from 100 ASD and 32 typically developing children of four different age groups. 1, 2, 3, 4 in the x-axis denotes 2-5, 6-10, 11-15, 16+ years age groups, respectively. The y-axis is the log transformed and median scaled metabolite level. Solid line indicates mean level of metabolites in ASD samples. Dotted line indicates mean level of metabolites in typically developing children samples. The plots show that the solid and dotted lines are closer for old age group and more separated for young age group.

FIG. 3 is a box plot illustrating the quantitation of 18 metabolites in plasma of 127 children with ASD ages 2-10 years (ASD) and 82 age-matched typical developing (TD) children. Both box plots and jitter plots of quantitation of each metabolite are shown. The black dots indicate the outliers.

FIG. 4 is a heat map of levels of 25 metabolites of 50 ASD and 16 typical developing children samples (Controls) of 2-5 and 6-10 years age groups. The metabolite level (median scaled only without log transformation) is scaled accordingly. Black and grey indicate high and low levels, respectively.

FIG. 5 at A presents a Principal component analysis (PCA) plot of global metabolome data from plasma of 100 ASD and 32 typically developing (TD) children of four different age groups. (PC—principal component). For data of all ages, the first principal component (PC1), which represents the largest variance, is among ages. At B is shown a Linear discriminant analysis (LDA) plot of metabolome data of 100 ASD and 32 TD plasma samples of four different age groups for first two linear discriminants. The samples were grouped with status (ASD or TD) and age (2-5, 6-10, 11-15, 16+ years).

FIG. 6 at A presents a PCA plot of plasma metabolome data of 53 ASD and 16 TD samples of age groups 2-5 and 6-10 years. The PC1 is between ASD and TD. At B is shown an LDA plot of plasma metabolome data of 53 ASD and 16 TD samples of age groups 2-5 and 6-10 years. The samples were grouped with status (ASD or TD) and age (2-5, 6-10 years) for LDA study.

FIG. 7 shows a linear discriminant analysis comparing the plasma metabolite concentrations of 136 children diagnosed with autism age 10 years and less with those of 92 age-matched typically developing children.

FIG. 8 provides a partition of the linear discriminant analysis comparing the plasma metabolite concentrations of 136 children diagnosed with autism ages 10 years or less with those of 92 age-matched typically developing children (as in FIG. 7) demonstrating values of typically developing children above and below the 90th centile and values of children diagnosed with ASD above and below the 10th centile.

FIG. 9 shows the partition of quantitative plasma concentration levels for seven different metabolites from 36 children diagnosed with ASD and 16 typically developing children. Values above and below 10th centile for ASD-diagnosed children are indicated by the solid line and values above and below 90th centile for typically developing children are indicated by the dashed line.

FIG. 10 provides a Principal component analysis (PCA) of plasma metabolite concentrations from 78 children, males and females, diagnosed with ASD ages 2-5 years (circles) and 32 gender and age-matched typically developing children (triangles).

### DETAILED DESCRIPTION

Reference now will be made to embodiments of the disclosure, examples of which are set forth below. Each example is provided by way of an explanation of the disclosure, not as a limitation of the disclosure. In fact, it will be apparent to those skilled in the art that various modifications and variations can be made in the disclosure without departing from the scope or spirit of the disclosure. For instance, features illustrated or described as one embodiment can be used on another embodiment to yield still a further embodiment. It is to be understood by one of ordinary skill in the art that the present discussion is a description of exemplary embodiments only, and is not intended as limiting the broader aspects of the present disclosure, which broader aspects are embodied in exemplary constructions.

As used herein, the terms “autism” and “autism spectrum disorder” (ASD) are used interchangeably to generally describe three of the five developmental disorders described in the Diagnostic and Statistical Manual, IVth Edition (DSM IV-TR): autistic disorder, Asperger disorder, and Pervasive Developmental Disorder Not Otherwise Specified (American Psychiatric Association 2000) and/or described in the Diagnostic and Statistical Manual Vth Edition (DSM-5) (American Psychiatric Association, 2013).

As used herein, the terms “affected” and “affected child” generally refer to a child with features of autism or ASD as defined by The American Psychiatric Association (2000, 2013). While males are more commonly affected with ASD, both males and females may be affected. Conversely, the terms “non-affected”, “non-affected child”, “typically developing (TD) or “normal control” refers to a child without features of autism or ASD as defined by The American Psychiatric Association (2000, 2013).

As used herein the terms “marker” or “autism marker”, “biomarker” or “autism biomarker”, or “autism metabolic biomarker” generally refers to a compound or a biochemical that can be used to directly or indirectly aid in diagnosis of an individual affected with autism.

As used herein, the term “test sample” generally refers to a biological material suspected of containing the metabolic biomarkers as described herein. The test sample may be derived from any biological source, such as a physiological fluid, including, blood, plasma, serum, interstitial fluid, saliva, cerebrospinal fluid, sweat, urine, ascites fluid, mucous, nasal fluid, sputum, peritoneal fluid, and so forth. The test sample may be used directly as obtained from the subject or following a pretreatment to modify the character of the sample. For example, such pretreatment may include isolating plasma from blood, collection and drying of blood on filter paper (dry blood spots) diluting viscous fluids, and so forth. Methods of pretreatment may also involve elution, filtration, precipitation, dilution, distillation, mixing, concentration, inactivation of interfering components, the addition of reagents, lysing, etc. Moreover, it may also be beneficial to modify a solid test sample to form a liquid medium or to release the analyte.

In general, the present disclosure is directed to methods for early detection of autism through analysis of the concentration levels of metabolic biomarkers in a test sample. More specifically, disclosed methods can be successfully utilized for the detection of ASD in children under the age of about 10 years. Beneficially, in one embodiment the disclosed methods can be utilized for pre-symptomatic and early symptomatic detection of autism in children.

The diagnosis methods can utilize relatively simple laboratory procedures that can be carried out with a test sample obtained from a subject of about 10 years or less or about 5 years or less in some embodiments. For instance, a subject can be from about 2 years of age to about 10 years of age, or from about 2 years of age to about 5 years of age in some embodiments. Beneficially, the early diagnosis capable by use of the methods can result in early treatments that can, e.g., improve speech development and social interaction. The methods can also provide pre-symptomatic screening for siblings or other relatives of affected individuals. This can prove beneficial as previously, such testing for a sibling of an affected individual generally had to wait for the sibling to reach approximately 3 years of age for evaluations. Of course, disclosed methods may also be used to identify children with ASD in families which do not have previously affected relatives.

Methods disclosed herein can be utilized as a quick and reliable screening procedure for ASD, as a diagnostic tool for ASD that can be utilized in conjunction with other traditional behavioral diagnostic procedures, and as a measure to monitor treatment for ASD. Moreover, disclosed methods can be utilized in providing a unifying model for the causal heterogeneity of ASD.

The methods generally include determining the concentration level in a test sample of two or more metabolites that have been discovered to be deterministic biomarkers of ASD and comparing those levels to levels in controls. Determination that the metabolite concentration levels in the test sample differ from the control levels by a significant amount can indicate that the subject is affected or will become affected with ASD.

While not wishing to be bound to any particular theory, it is believed that during the initial months after delivery, the metabolism of children with autism fails to follow the same trajectory as metabolism of typically developing children. This deviation from typical development is believed to result in a state of metabolic anarchy in children with autism which is manifest by the levels of a number of metabolites deviating significantly from the levels of these metabolites in typically developing children. The discriminating metabolites represent diverse metabolic pathways including, among others, amino acid, fatty acid, inflammatory, energy and neurotransmitter pathways. In turn, the disturbances in these pathways are reliable indicators of the neurobehavioral manifestations in children with autism.

Surprisingly, it has been found that the disturbance in metabolism as indicated by the levels of the discriminating metabolites decreases for some or increases for others over time, with the levels of the biomarker metabolites described herein transitioning to a typical or near typical profile with increasing age. Thus, it is believed that the child with autism may at some age acquire the metabolic profile of typically developing children. However, these children generally retain all or some lesser measure of the neurobehavioral attributes of autism.

This transient nature of the metabolic disturbance suggests not only that disclosed diagnostic methods are generally limited to affected individuals that have not yet transitioned to a more typical metabolite profile for the indicated metabolites, but also that the window of metabolite-based therapeutic opportunity may also be limited to this pre-transition period. For instance, metabolite-based therapy (embodiments of which are described further herein) can be carried out during the periods of speech acquisition, formation of social intercourse, and inhibition of inappropriate actions/reactions/responses for affected individuals of about 10 years or younger, or about 5 years or younger in some embodiments, for instance from about 2 years old to about 10 years old, or from about 2 years old to about 5 years old in some embodiments.

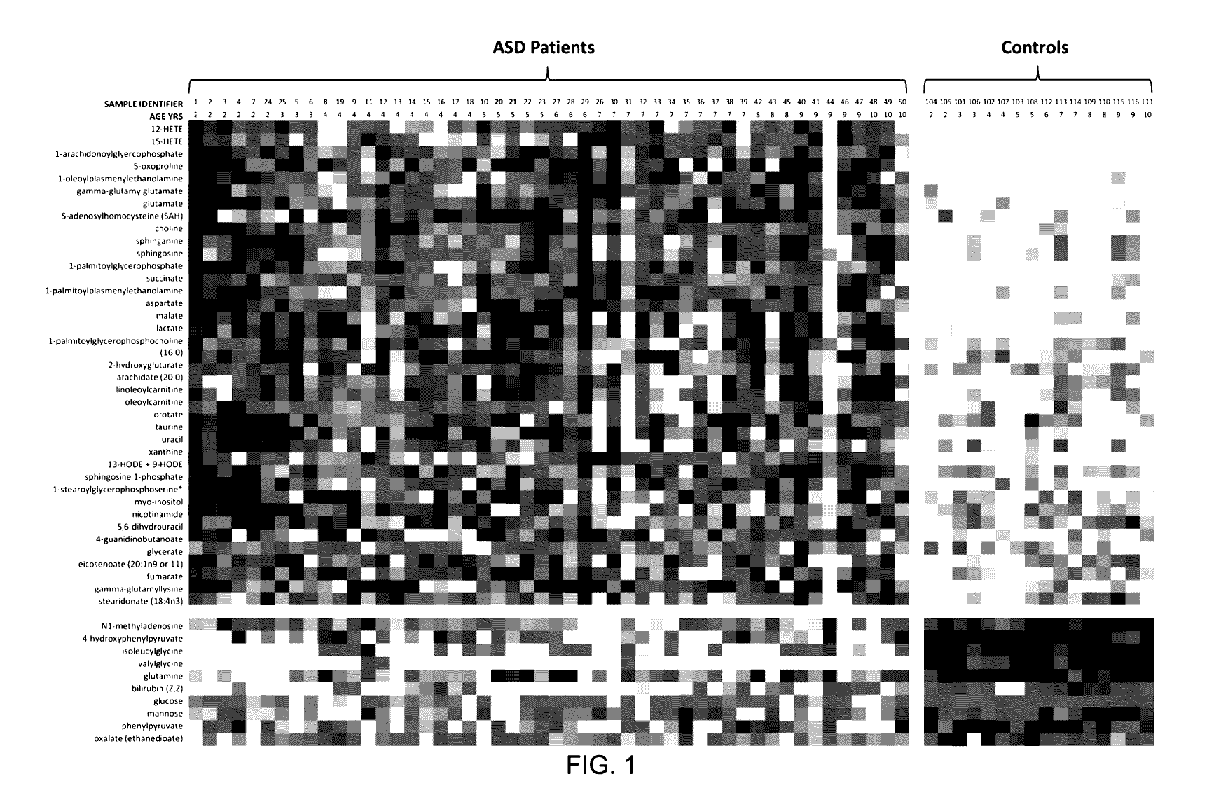

Table 1 presented below provides a list of 48 different biomarker metabolites the concentrations of which have been found to deviate significantly in children diagnosed with autism. The up and down arrows in the second column of the table indicate whether the metabolite is found in high (↑) or low (↓) concentrations in ASD diagnosed children as compared to controls. FIG. 1 is a heat map (converted form a typical color heat map) showing the plasma concentration levels of these 48 metabolites including 38 metabolites that are increased and 10 metabolites that are decreased in 50 ASD diagnosed children ranging in age from 2 years to 10 years in comparison to levels of these metabolites in 16 age-matched children developing typically (controls). The metabolite level (median scaled only without log transformation) was scaled accordingly. Darker and lighter indicate higher and lower levels, respectively.

TABLE 1

| | | | SUPER | |
|---|---|---|---|---|
| | ↑/↓ | Metabolite/Alias | PATHWAY | SUB PATHWAY |
| 1 | ↓ | glucose | Carbohydrate | Glycolysis, Gluconeogenesis, and |
| | | | | Pyruvate |
| | | | | Metabolism |
| 2 | ↑ | lactate | Carbohydrate | Glycolysis, Gluconeogenesis, and |
| | | | | Pyruvate |
| | | | | Metabolism |
| 3 | ↑ | glycerate | Carbohydrate | Glycolysis, Gluconeogenesis, and |
| | | | | Pyruvate |
| | | | | Metabolism |
| 4 | ↓ | mannose | Carbohydrate | Fructose, Mannose and Galactose |
| | | | | Metabolism |
| 5 | ↑ | fumarate | Energy | TCA Cycle |
| 6 | ↑ | malate | Energy | TCA Cycle |
| 7 | ↑ | succinate | Energy | TCA Cycle |
| 8 | ↑ | 12-HETE | Lipid | Eicosanoid |
| 9 | ↑ | 15-HETE | Lipid | Eicosanoid |
| 10 | ↑ | choline | Lipid | Phospholipid Metabolism |
| 11 | ↑ | 1-arachidonoylglyercophosphate/ | Lipid | Lysolipid |
| | | 1-arachidonoyl-GPA (20:4) | | |
| 12 | ↑ | 1-oleoylplasmenylethanolamine/ | Lipid | Lysolipid |
| | | 1-(1-enyl-oleoyl)-GPE | | |
| 13 | ↑ | 1-palmitoylglycerophosphate/ | Lipid | Lysolipid |
| | | 1-palmitoyl-GPA (16:0) | | |
| 14 | ↑ | 1-palmitoylglycerophosphocholine | Lipid | Lysolipid |
| | | (16:0)/1-palmitoyl-GPC (16:0) | | |
| 15 | ↑ | 1- | Lipid | Lysolipid |
| | | palmitoylplasmenylethanolamine/ | | |
| | | 1-(1-enyl-palmitoyl)-GPE | | |
| 16 | ↑ | 1-stearoylglycerophosphoserine/ | Lipid | Lysolipid |
| | | 1-stearoyl-GPS (18:0) | | |
| 17 | ↑ | 13-HODE + 9-HODE | Lipid | Fatty Acid, Monohydroxy |
| 18 | ↑ | 2-hydroxyglutarate | Lipid | Fatty Acid, Dicarboxylate |
| 19 | ↑ | arachidate (20:0) | Lipid | Long Chain Fatty Acid |
| 20 | ↑ | eicosenoate (20:1) | Lipid | Long Chain Fatty Acid |
| 21 | ↑ | linoleoylcarnitine | Lipid | Fatty Acid Metabolism(Acyl Carnitine) |
| 22 | ↑ | oleoylcarnitine | Lipid | Fatty Acid Metabolism(Acyl Carnitine) |
| 23 | ↑ | myo-inositol | Lipid | Inositol Metabolism |
| 24 | ↑ | sphinganine | Lipid | Sphingolipid Metabolism |
| 25 | ↑ | sphingosine (SPH) | Lipid | Sphingolipid Metabolism |
| 26 | ↑ | sphingosine 1-phosphate | Lipid | Sphingolipid Metabolism |
| 27 | ↑ | stearidonate (18:4n3) | Lipid | Polyunsaturated Fatty Acid (n3 and |
| | | | | n6) |
| 28 | ↑ | 5-oxoproline | Amino Acid | Glutathione Metabolism |
| 29 | ↑ | aspartate | Amino Acid | Alanine and Aspartate Metabolism |
| 30 | ↑ | glutamate | Amino Acid | Glutamate Metabolism |
| 31 | ↓ | glutamine | Amino Acid | Glutamate Metabolism |
| 32 | ↑ | S-adenosylhomocysteine (SAH) | Amino Acid | Methionine, Cysteine, SAM and |
| | | | | Taurine |
| | | | | Metabolism |
| 33 | ↑ | taurine | Amino Acid | Methionine, Cysteine, SAM and |
| | | | | Taurine |
| | | | | Metabolism |
| 34 | ↑ | 4-guanidinobutanoate | Amino Acid | Guanidino and Acetamido Metabolism |
| 35 | ↓ | 4-hydroxyphenylpyruvate | Amino Acid | Phenylalanine and Tyrosine |
| | | | | Metabolism |
| 36 | ↓ | phenylpyruvate | Amino Acid | Phenylalanine and Tyrosine |
| | | | | Metabolism |
| 37 | ↑ | 5,6-dihydrouracil | Nucleotide | Pyrimidine Metabolism, Uracil |
| | | | | containing |
| 38 | ↑ | uracil | Nucleotide | Pyrimidine Metabolism, Uracil |
| | | | | containing |
| 39 | ↑ | orotate | Nucleotide | Pyrimidine Metabolism, Orotate |
| | | | | containing |
| 40 | ↓ | N1-methyladenosine | Nucleotide | Purine Metabolism, Adenine |
| | | | | containing |
| 41 | ↑ | xanthine | Nucleotide | Purine Metabolism, |
| | | | | (Hypo)Xanthine/Inosine containing |
| 42 | ↑ | gamma-glutamylglutamate | Peptide | Gamma-glutamyl Amino Acid |
| 43 | ↑ | gamma-glutamyllysine | Peptide | Gamma-glutamyl Amino Acid |
| 44 | ↓ | valylglycine | Peptide | Dipeptide |
| 45 | ↓ | isoleucylglycine | Peptide | Dipeptide |
| 46 | ↑ | nicotinamide | Cofactors and | Nicotinate and Nicotinamide |
| | | | Vitamins | Metabolism |
| 47 | ↓ | bilirubin (Z,Z) | Cofactors and | Hemoglobin and Porphyrin Metabolism |
| | | | Vitamins | |
| 48 | ↓ | oxalate (ethanedioate) | Cofactors and | Ascorbate and Aldarate Metabolism |
| | | | Vitamins | |

As can be seen by reference to Table 1 and FIG. 1, the biomarker metabolites include those that are significantly increased as well as metabolites that are significantly decreased in children diagnosed with ASD as compared to levels of the same metabolites in typically developing children. In addition, these biomarker metabolites can be found in a large number of different metabolic pathways, as indicated.

In one embodiment, a testing protocol can include the determination in a sample of the concentration level for all 48 metabolites listed in Table 1. Determination that a majority of the metabolites, e.g., 24 or more of the metabolites, 30 or more of the metabolites, or 40 or more of the metabolites in some embodiments, have a concentration in the sample that is significantly different (either higher or lower) from a predetermined control level and/or outside of a predetermined control range of the metabolite level can indicate that the subject is affected with ASD.

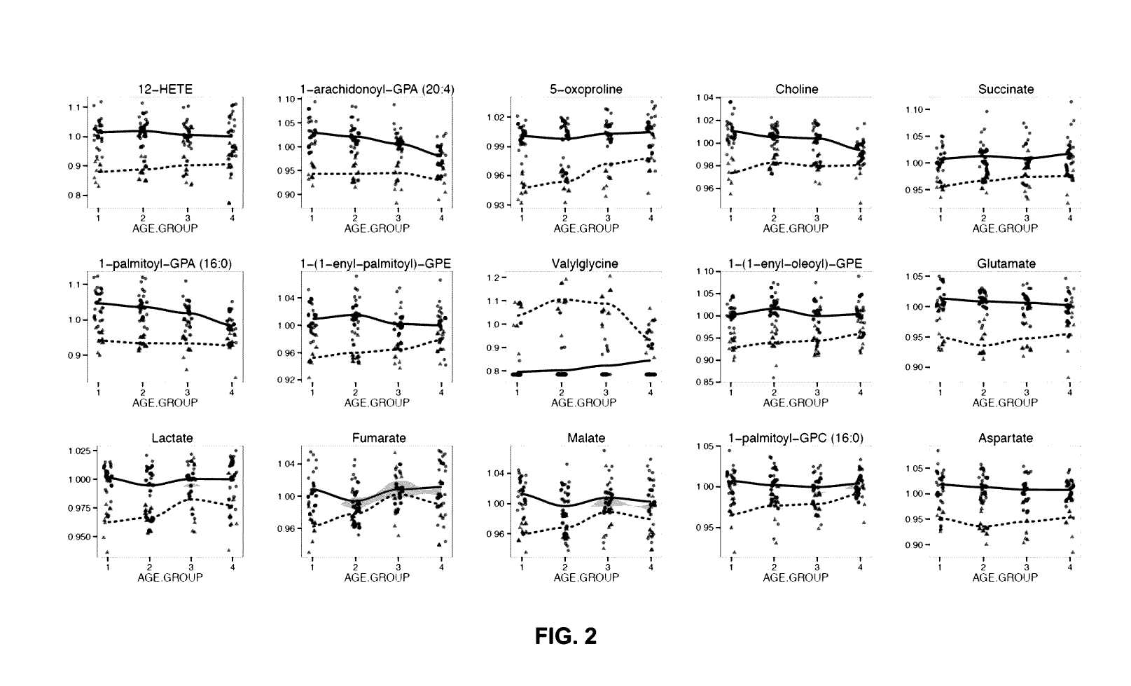

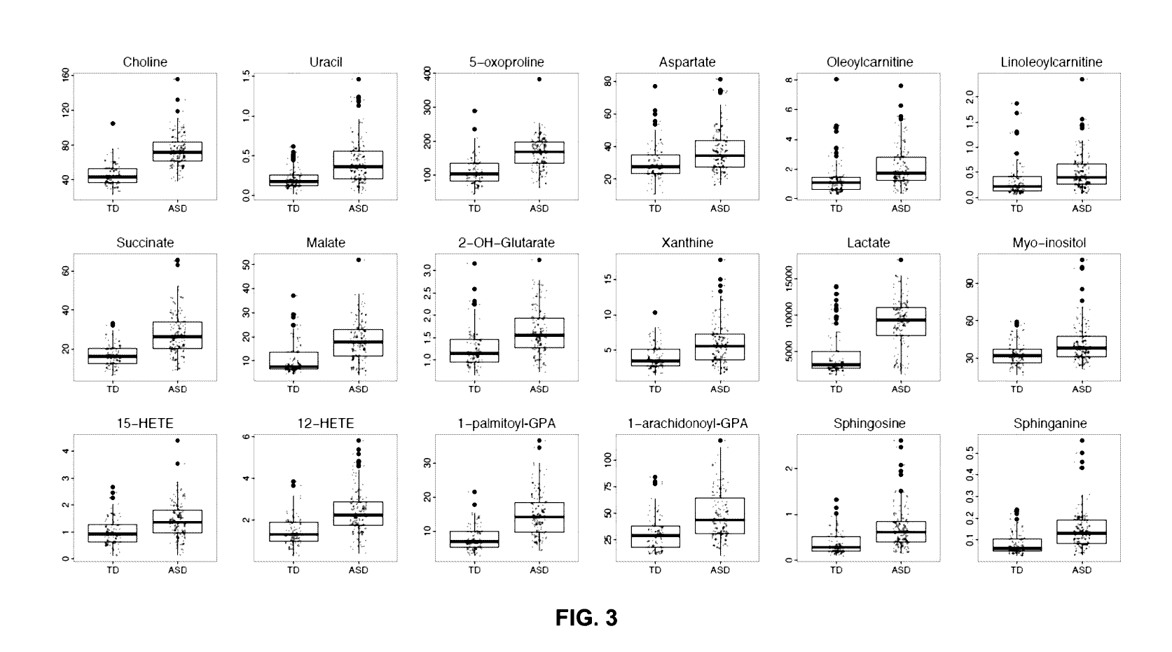

Determination of the control level and/or control range for a metabolite can be carried out according to standard practice. In general, the control metabolite concentration for each of the metabolites to be examined in a testing protocol can be developed from data obtained from a control group comprising non-affected age matched individuals. A predetermined control range can be, for example, within one standard deviation of the average value of that particular metabolite level found in the control group. One method of comparing the concentration level of a particular metabolite of an individual to a predetermined control range of concentration levels for that metabolite involves plotting the value of the test subject's metabolite level against a scatterplot of the concentration levels of that particular metabolite taken from a plurality of non-affected persons by the same methods and using the same sample type (FIG. 2 and FIG. 3). For example, a plot can be used to create a chart having on the X-axis the age of the person from which the sample was obtained and on the Y-axis the concentration level of the metabolite being examined. When analyzing the resulting scatterplot chart, if the metabolite concentration level of the test subject falls above or below the metabolite concentration levels of non-affected persons, and this result is repeated for one or more additional metabolites from the table, then the individual may be affected with autism. By way of example, if the metabolite concentration levels of the tested individual is statistically different (e.g., P value of <0.05) from the average metabolite concentration levels for the tested metabolites of the above table, then the individual may be affected with ASD.

When conducting a comparison of the concentration level of a particular metabolite from the test sample of an individual against concentration levels of that metabolite taken from a control group of non-affected persons, the number of controls (non-affected persons) may vary, as is generally known in the art. However, in order to be of increased value, a statistically significant number of controls are generally utilized. For instance, at least about 2 controls can be utilized. In other embodiments, more controls such as about 10, about 25, about 40 or about 100 controls can be utilized to create a suitable control level or control range for a particular metabolite.

Table 2 below illustrates the significant quantitative differences found in a representative sample of 7 of the biomarker metabolites from Table 1 between children diagnosed with ASD and typically developing children of a control sample. The 7 biomarker metabolites in Table 2 include 12-HETE, sphingosine (SPH), choline, aspartate, lactate, malate, and succinate. The data were determined through examination of the metabolite levels in 36 children diagnosed with ASD (age 2-10 years) and in 16 typically developing children (age-matched). Concentrations are provided as μM. The analysis was performed by tandem mass spectrometry.

TABLE 2

| Sample ID | 12-HETE | SPH | choline | aspartate | lactate | malate | succinate |
|---|---|---|---|---|---|---|---|
| ASD_1 | 10.5 | 135678.7 | 41.0 | 29.8 | 7824.1 | 16.1 | 30.3 |
| ASD_2 | 5.5 | 76301.9 | 48.3 | 29.9 | 10283.1 | 12.9 | 27.8 |
| ASD_3 | 8.9 | 86337.4 | 37.5 | 49.1 | 6732.1 | 13.8 | 22.6 |
| ASD_4 | 3.7 | 254718.1 | 31.7 | 28.4 | 9609.5 | 12.5 | 22.2 |
| ASD_5 | 4.0 | 156846.8 | 25.3 | 14.8 | 12284.0 | 17.9 | 21.2 |
| ASD_6 | 3.9 | 97905.1 | 19.7 | 20.4 | 6707.9 | 8.5 | 14.9 |
| ASD_7 | 4.4 | 118932.5 | 26.2 | 20.3 | 9464.9 | 12.1 | 39.5 |
| ASD_8 | 1.6 | 51021.3 | 25.2 | 21.5 | 11595.0 | 21.4 | 17.7 |
| ASD_9 | 3.1 | 48067.7 | 25.3 | 19.1 | 10359.2 | 12.7 | 16.4 |
| ASD_10 | 7.0 | 39354.4 | 29.2 | 25.5 | 4821.4 | 9.8 | 24.0 |
| ASD_11 | 3.4 | 30584.8 | 19.4 | 13.0 | 3916.1 | 6.4 | 10.7 |
| ASD_12 | 3.5 | 63083.4 | 39.9 | 23.3 | 6786.2 | 11.3 | 24.0 |
| ASD_13 | 2.8 | 60179.1 | 36.4 | 21.2 | 7300.2 | 7.4 | 22.6 |
| ASD_14 | 3.6 | 60418.0 | 26.7 | 18.6 | 7180.0 | 10.2 | 21.0 |
| ASD_15 | 3.1 | 43291.5 | 21.4 | 17.7 | 8087.1 | 11.3 | 21.2 |
| ASD_16 | 2.1 | 57906.3 | 25.7 | 14.1 | 7794.4 | 10.8 | 13.1 |
| ASD_17 | 4.4 | 66478.5 | 24.8 | 16.5 | 8202.6 | 9.7 | 16.9 |
| ASD_18 | 2.9 | 36792.3 | 22.9 | 9.7 | 12160.7 | 18.4 | 19.9 |
| ASD_19 | 3.4 | 15777.6 | 11.7 | 12.6 | 2998.5 | 3.7 | 7.5 |
| ASD_20 | 5.9 | 40102.0 | 36.6 | 25.4 | 10992.7 | 14.2 | 28.6 |
| ASD_21 | 3.8 | 28742.1 | 26.0 | 18.2 | 7519.0 | 9.5 | 16.3 |
| ASD_22 | 4.4 | 60512.3 | 32.6 | 29.2 | 11031.8 | 15.6 | 37.0 |
| ASD_23 | 10.3 | 60498.8 | 33.0 | 23.6 | 9135.0 | 11.8 | 49.8 |
| ASD_24 | 3.7 | 19759.2 | 21.6 | 16.9 | 3201.9 | 4.9 | 12.2 |
| ASD_25 | 3.2 | 52810.4 | 24.0 | 20.3 | 11473.4 | 14.2 | 18.8 |
| ASD_26 | 4.8 | 51653.7 | 22.0 | 21.8 | 5724.5 | 8.9 | 16.6 |
| ASD_27 | 2.0 | 63139.9 | 33.7 | 19.9 | 8744.3 | 13.1 | 29.8 |
| ASD_28 | 3.2 | 25726.8 | 26.8 | 15.9 | 2898.0 | 6.1 | 20.1 |
| ASD_29 | 2.6 | 56757.2 | 23.3 | 21.3 | 8473.0 | 8.0 | 17.2 |
| ASD_30 | 4.0 | 16622.2 | 23.4 | 16.6 | 3056.5 | 5.5 | 12.8 |
| ASD_31 | 6.9 | 84611.3 | 28.3 | 33.3 | 8088.1 | 10.4 | 17.6 |
| ASD_32 | 2.5 | 62416.8 | 23.6 | 28.5 | 8041.4 | 9.2 | 14.2 |
| ASD_33 | 6.6 | 60255.0 | 31.4 | 34.4 | 10258.2 | 13.2 | 23.1 |
| ASD_34 | 5.4 | 24650.3 | 27.1 | 16.4 | 10313.1 | 12.7 | 25.2 |
| ASD_35 | 3.9 | 40991.2 | 34.6 | 21.6 | 11245.7 | 13.6 | 26.5 |
| ASD_36 | 5.4 | 37549.1 | 27.4 | 19.0 | 2813.3 | 5.9 | 21.7 |
| | 160.3 | 2286473.6 | 1013.7 | 787.8 | 287116.8 | 403.9 | 780.8 |
| Mean | 4.5 | 63513.2 | 28.2 | 21.9 | 7975.5 | 11.2 | 21.7 |
| 10th | 2.6 | 25188.6 | 21.5 | 14.5 | 3129.2 | 6.0 | 13.0 |
| Centile | | | | | | | |
| Control_1 | 1.0 | 12892.8 | 19.3 | 11.1 | 3260.9 | 5.1 | 9.0 |
| Control_2 | 0.7 | 11084.3 | 16.8 | 9.9 | 3295.6 | 5.2 | 5.6 |
| Control_3 | 1.3 | 9118.2 | 11.0 | 8.8 | 3070.0 | 5.1 | 7.0 |
| Control_4 | 1.7 | 6279.6 | 13.0 | 13.4 | 1537.9 | 3.8 | 10.1 |
| Control_5 | 1.1 | 8531.2 | 15.1 | 12.2 | 3966.3 | 7.0 | 7.4 |
| Control_6 | 2.1 | 18294.2 | 17.0 | 12.9 | 3154.4 | 5.6 | 16.6 |
| Control_7 | 2.7 | 18317.7 | 16.6 | 14.6 | 3572.1 | 5.8 | 7.4 |
| Control_8 | 1.8 | 7295.7 | 17.1 | 16.9 | 2022.8 | 5.4 | 12.1 |
| Control_9 | 1.9 | 11736.5 | 17.2 | 7.0 | 2118.9 | 5.0 | 11.4 |
| Control_10 | 2.4 | 14429.4 | 22.4 | 10.8 | 3106.1 | 4.4 | 11.9 |
| Control_11 | 2.1 | 10633.2 | 15.0 | 10.6 | 3042.2 | 6.5 | 10.9 |
| Control_12 | 1.5 | 7957.0 | 15.7 | 8.3 | 2045.1 | 5.0 | 11.2 |
| Control_13 | 1.4 | 19162.5 | 16.3 | 15.0 | 3553.2 | 7.2 | 9.7 |
| Control_14 | 1.2 | 4021.9 | 16.2 | 9.6 | 2596.3 | 6.2 | 10.4 |
| Control_15 | 2.5 | 24934.6 | 14.5 | 18.0 | 4676.4 | 6.4 | 11.1 |
| Control_16 | 2.4 | 12890.8 | 15.1 | 8.5 | 3372.9 | 8.2 | 14.6 |
| | 27.6 | 197579.5 | 258.1 | 187.5 | 48390.8 | 91.9 | 166.6 |
| Mean | 1.7 | 12348.7 | 16.1 | 11.7 | 3024.4 | 5.7 | 10.4 |
| 90th | 2.5 | 18740.1 | 18.3 | 15.9 | 3769.2 | 7.1 | 13.4 |
| Centile | | | | | | | |

As can be seen in Table 2, the mean value for the plasma concentrations of these metabolites in the ASD children is about 50% or more different (either higher or lower, depending upon the metabolite) from the mean value for the plasma concentrations of same metabolite in age-matched typically developing children. In one embodiment, the difference between a tested concentration and a control concentration can be a standard for determining a significantly different concentration level of a biomarker metabolite in a tested subject. For instance, if the concentration level of a metabolite in a test sample differs from a control level for that metabolite by about 30% or greater, about 50% or greater, about 70% or greater, about 90% or greater, or about 100% or greater, then the concentration level of that metabolite can be said to be significantly different from that of the control level. The difference can be determined according to standard practice, i.e., the percentage difference between the tested concentration (Cₜₑₛₜ) and the control concentration (C_{control}) can be determined according to the following equation:

Percentage difference=100×(|C_{control}−Cₜₑₛₜ|)/Cₜₑₛₜ

Of course, other standards for the significant difference, such as one standard deviation from a control value, outside of a control value range, and so forth as discussed above, may alternatively be utilized.

According to another embodiment, the ratio of concentrations of two metabolites in a test subject can be compared to the ratio of the control values for the same metabolites, e.g., the concentrations of the same metabolites in non-affected age-matched individuals and the comparison can be utilized to achieve equal or greater discrimination between the two populations. For example, the concentrations of metabolite 1 (M₁) and metabolite 2 (M₂) can be determined for a test subject and the ratio of these concentrations, i.e., ([M₁]/[M₂])ₜₑₛₜ can be compared to the same ratio of control values, i.e., ([M₁]/[M₂])_{control}). Thus, the test value can be: ([M₁]/[M₂])ₜₑₛₜ/([M₁]/[M₂])_{control}. When this test value difference is about 30% or greater, i.e.,

([M₁]/[M₂])ₜₑₛₜ/([M₁]/[M₂])_{control}≧0.30)

the result can signify a significant difference in metabolite concentration in a test sample.

A testing protocol can include comparing the test value for each two metabolites of the plurality of the biomarker metabolites examined in a sample or for a subset of all of the possible pairings of the biomarker metabolites examined in a sample. For instance, a test value as defined above can be obtained for each possible pair of biomarker metabolites in a test sample. If any one of those pairings provides a test value difference that is about 30% or greater, then the individual may be affected with ASD. For example, if 2 or more of those test values, 5 or more of those test values, or 10 or more of those test value differences are about 30% or greater, then the individual may be affected with ASD.

Methods as disclosed herein need not examine the concentration levels of all 48 biomarker metabolites. According to one embodiment, a subset of the 48 biomarker metabolites provided above may be examined quantitatively to discriminate children with ASD from typically developing children age 10 years and younger. According to this embodiment, a determination that all of the biomarker metabolites of the subset from the subject's test sample are at a concentration level that significantly differs from a control level or is outside of a control range for each metabolite can be an indication that the subject is affected with ASD. In general, a subset according to this embodiment can include at least one of 12-HETE and 15-HETE and can also include one of sphingosine and choline.

For illustrative purposes, one such subset of the biomarker metabolites can include 12-HETE and sphingosine. According to one embodiment, these two metabolite levels in test subjects can be quantitated and used to discriminate between children with ASD and typically developing children (TD) age 10 years and younger. Utilization of only these two metabolites in a testing protocol can be predictive of ASD with greater than about 90% predictive ability. Of course a subset testing protocol is not limited to use of only these two metabolites, and other metabolites of the above provided table can be utilized, optionally in conjunction with these two metabolites. For example, in one embodiment, the concentration level for 12-HETE and sphingosine can be utilized in conjunction with two or more additional of the 48 biomarker metabolites provided above. According to this embodiment, a finding that the concentrations of both 12-HETE and sphingosine in a test sample are significantly different as compared to their control levels in combination with a finding that the concentrations of one or more of the additional metabolites tested in the test sample are significantly different as compared to their control levels can signify that the subject is affected with ASD.

Another two metabolite subset as may be highly predictive of ASD includes both 12-HETE and choline. For instance, the levels in test subjects of both of these biomarker metabolites may be quantitated and a finding that both of these metabolites are present in a test sample in a significantly different amount as compared to the control levels can indicate that the subject is affected with ASD. Thus these two biomarker metabolites may be utilized alone or in conjunction with other biomarker metabolites of the above table to discriminate children with ASD from typically developing children age 10 years and younger, generally with greater than about 90% predictive ability.

A three metabolite subset of the biomarker metabolites can include either 12-HETE or 15-HETE in conjunction with choline and aspartate. Either of these three metabolite subsets (12-HETE, choline, and aspartate or 15-HETE, choline, and aspartate) may be quantitatively measured from a test sample and their concentrations utilized either alone or in conjunction with one or more additional biomarkers of the 48 biomarker metabolites to discriminate children with ASD from typically developing children (TD) age 10 years and younger, generally with greater than about 90% predictive ability.

Four metabolite subsets of the biomarker metabolites can include choline in conjunction with one of lactate or glucose, one of succinate or malate, and one of 5-oxoproline or aspartate. The concentrations of the members of any of these four metabolite subsets may be quantitatively measured from a test sample and their concentrations utilized either alone or in conjunction with one or more additional biomarkers of the 48 biomarker metabolites to discriminate children with ASD from typically developing children (TD) age 10 years and younger, generally with greater than about 90% predictive ability. In general, when utilizing a subset of four or more of the 48 metabolite set, a finding that two or more of the metabolites in the subset have a concentration that significantly differs from that of a control level can indicate that the subject is affected with ASD.

A five metabolite subset as may be utilized to diagnose autism can include 4-hydroxyphenylpyruvate, 12-HETE, choline, sphingosine, and malate. According to one embodiment, these five metabolites of the 48 biomarker metabolites described above can be quantitatively measured and their concentrations utilized either alone or in conjunction with one or more additional biomarkers of the 48 biomarker metabolites to discriminate children with ASD from typically developing children (TD) age 10 years and younger, generally with greater than about 90% predictive ability. For instance, a determination that a test sample includes significantly different levels of two or more of the five metabolites of the subset as compared to control levels can indicate that the subject is affected with ASD.

A seven metabolite subset as may be utilized to diagnose autism can include one of either oleoylcarnitine or linoleoylcarnitine and one of either isoleucylglycine or valylglycine together with choline, 5-oxoproline, succinate, 4-hydroxyphenylpyruvate, and sphingosine. According to one embodiment, the members of one of these seven metabolite subsets can be quantitatively measured and their concentrations utilized either alone or in conjunction with one or more additional biomarkers of the 48 biomarker metabolites to discriminate children with ASD from typically developing children (TD) age 10 years and younger, generally with greater than about 90% predictive ability. For instance, a determination that a test sample includes significantly different levels of two or more of the seven metabolites of the subset as compared to control levels can indicate that the subject is affected with ASD.

Eight metabolite subsets as may be utilized to diagnose autism can include either 12-HETE or 15-HETE in conjunction with 5-oxoproline, choline, succinate, 1-palmitoylglycerophosphate, lactate, malate, and 4-hydroxyphenylpyruvate. According to one embodiment one of these eight metabolite subsets may be quantitatively measured and their concentrations utilized either alone or in conjunction with one or more additional biomarkers of the 48 biomarker metabolites to discriminate children with ASD from typically developing children (TD) age 10 years and younger, generally with greater than about 90% predictive ability. For instance, a determination that a test sample includes significantly different levels of two or more of the eight metabolites as compared to control levels can indicate that the subject is affected with ASD.

A 15 metabolite subset of the 48 biomarkers detailed above can include choline, 1-arachidonoylglycercophosphate, 5-oxoproline, succinate, lactate, fumarate, malate, 1-palmitoylglycerophosphocholine (16:0), aspartate, gamma-glutamylglutamate, isoleucylglycine, sphingosine, 4-hydroxyphenylpyruvate, glucose, and uracil. In one embodiment, a determination that a test sample includes significantly different levels of two or more of these 15 metabolites as compared to control levels can indicate that the subject is affected with ASD.

Another 15 metabolite subset of the 48 biomarkers detailed above can include 12-HETE, 1-arachidonoylglycercophosphate, 5-oxoproline, choline, succinate, 1-palmitoylglycerophosphate (16:0), 1-palmitoylplasmenylethanolamine, valylglycine, 1-oleoylplasmenylethanolamine, glutamate, lactate, fumarate, malate, 1-palmitoylglycerophosphocholine, aspartate. In one embodiment, a determination that a test sample includes significantly different levels of two or more of these 15 metabolites as compared to control levels can indicate that the subject is affected with ASD. FIG. 2 illustrates a jitter plot of levels of these 15 metabolites in plasma from 100 ASD and 32 typically developing children of four different age groups. 1, 2, 3, 4 in the x-axis denotes 2-5, 6-10, 11-15, 16+ years age groups, respectively. The y-axis is the log transformed and median scaled metabolite level. Solid line indicates mean level of metabolites in ASD samples. Dotted line indicates mean level of metabolites in typically developing children samples. The plots show that the solid and dotted lines are closer for old age group and more separated for young age group.

An 18 metabolite subset of the 48 biomarkers detailed above can include choline, uracil, 5-oxoproline, aspartate, oleocarnitine, linoleoylcarnitine, succinate, malate, 2-hydroxyglutarate, xanthine, lactate, myo-inositol, 15-HETE, 12-HETE, 1-palmitoylglycerophosphocholine, 1-arachidonoylglycercophosphate, sphingosine, and sphinganine. In one embodiment, a determination that a test sample includes significantly different levels of two or more of these 15 metabolites as compared to control levels can indicate that the subject is affected with ASD. FIG. 3 is a box plot illustrating the quantitation of these 18 metabolites in plasma of 127 children with ASD ages 2-10 years (ASD) and 82 age-matched typical developing (TD) children. Both box plots and jitter plots of quantitation of each metabolite are shown. The black dots indicate the outliers.

Another subset of the 48 biomarker metabolites can include the following 25 biomarker metabolites: 12-HETE, 15-HETE, 1-arachidonoylglyercophosphate, 5-oxoproline, 1-oleoylplasmenylethanolamine, gamma-glutamylglutamate, 4-guanidinobutanoate, glutamate, S-adenosylhomocysteine, glycerate, myo-inositol, choline, sphinganine, sphingosine, 1-palmitoylglycerophosphate, succinate, 1-palmitoylplasmenylethanolamine, aspartate, malate, lactate, glutamine, N1-methyladenosine, 4-hydroxyphenylpyruvate, isoleucylglycine, and valylglycine.

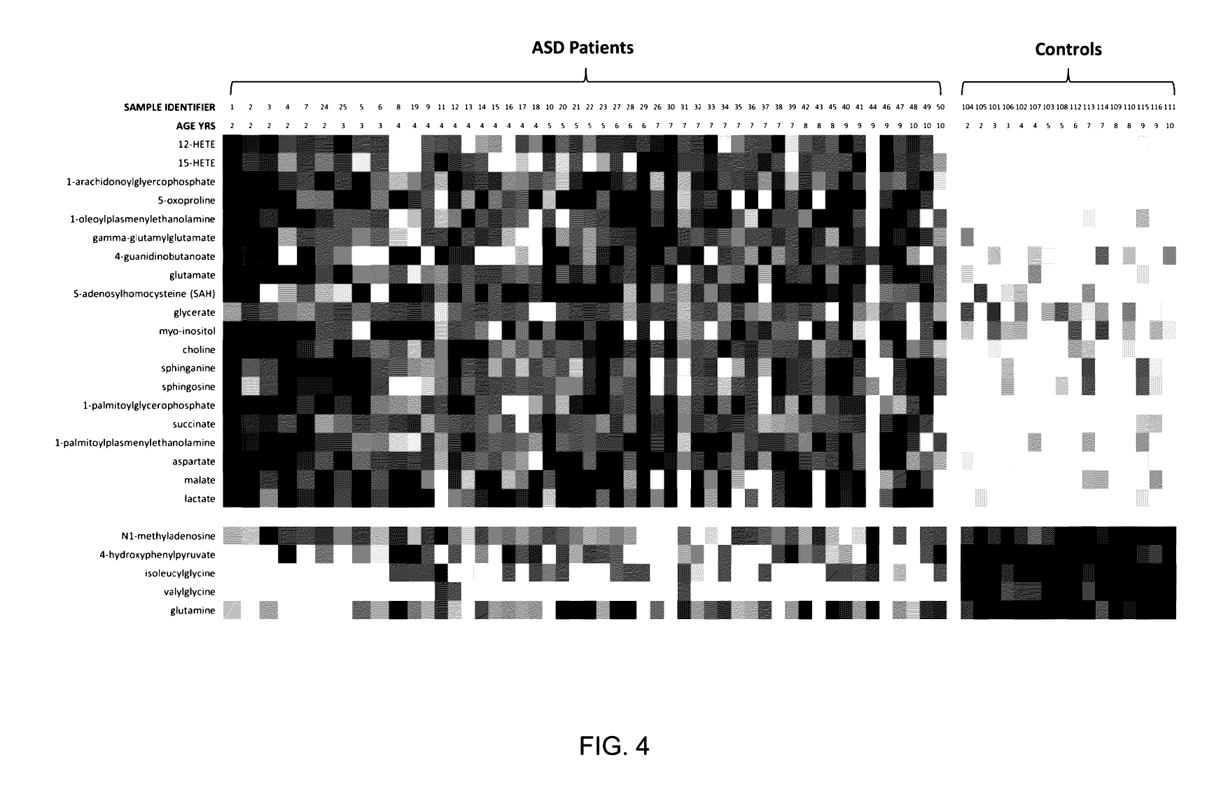

FIG. 4 is a heat map (converted from a typical color heat map) showing the concentration of these 25 biomarker metabolites in 50 children diagnosed with ASD age 10 years or less and comparison of those levels to the plasma concentrations of the same metabolites in typically developing children (16 age-matched children age 10 years or less). As can be seen in FIG. 4, the concentration of these 25 metabolites strongly discriminates between the ASD-diagnosed children and the typically developing children, with 20 of the metabolites being increased in concentration and 5 of the metabolites being decreased in concentration in the ASD-diagnosed children as compared to the typically developing children.

According to one embodiment a diagnosis method can include determining the concentration levels for the subset of these 25 biomarker metabolites and comparing those levels to control concentration levels for each of these biomarker metabolites. A finding that a plurality, e.g., 2 or more, about 3 or more, about 5 or more, about 10 or more, about 12 or more, about 15 or more, about 20 or more or all 25 of the tested biomarker metabolite concentrations are significantly different from the control concentrations can signify that the subject is affected with ASD.

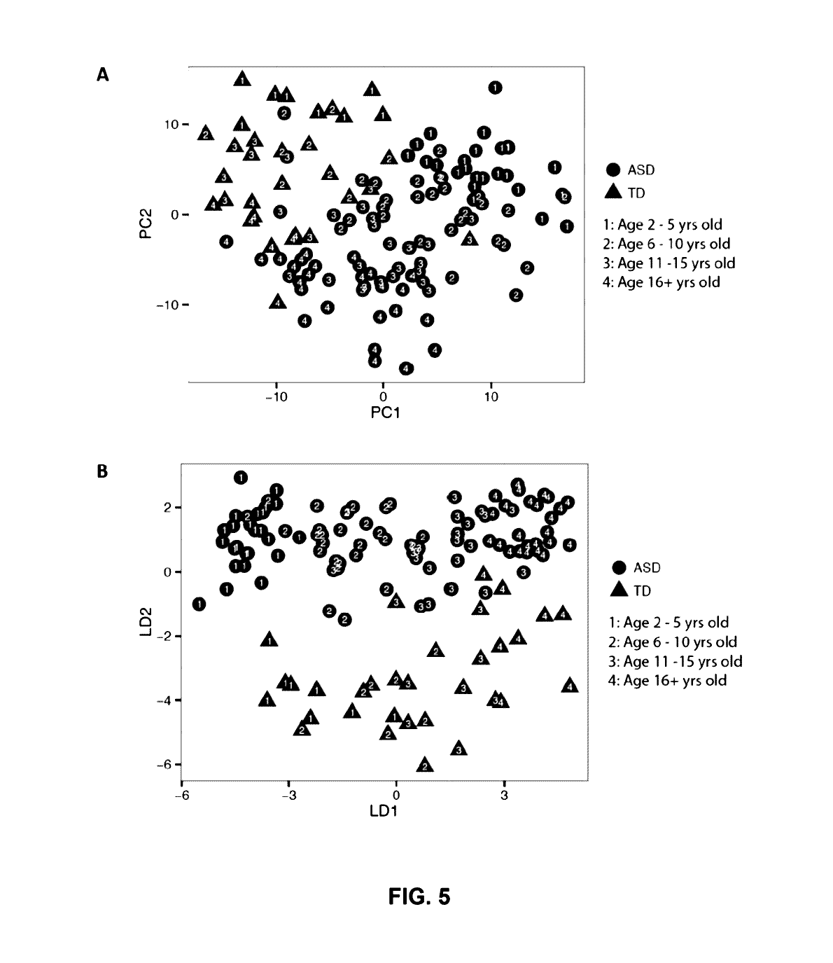

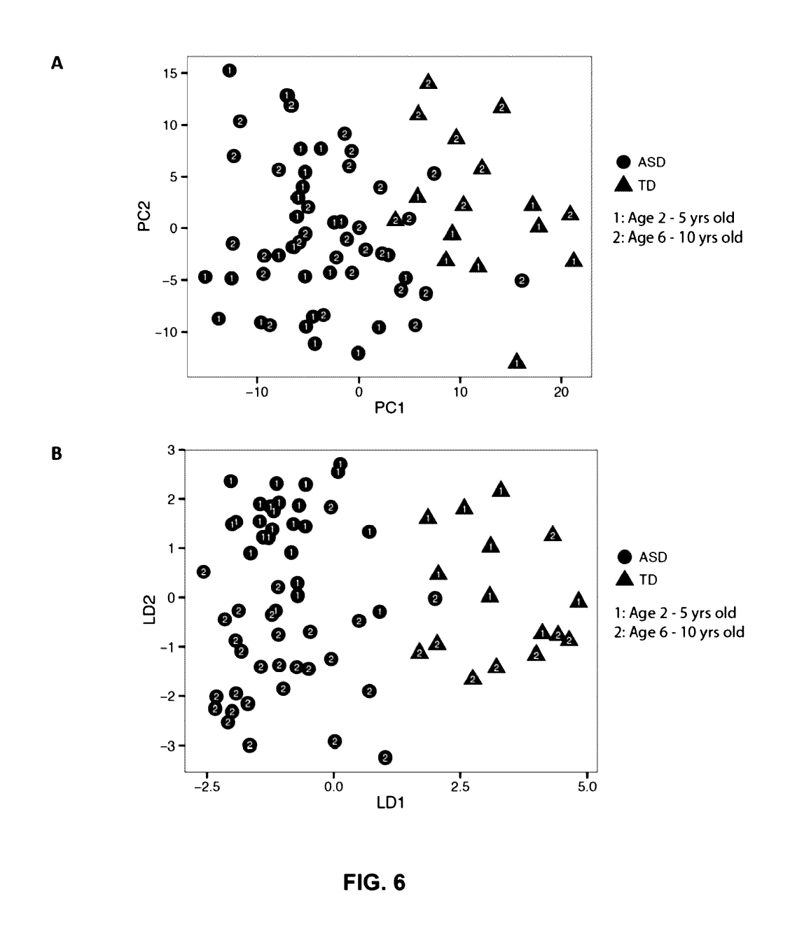

The differentials in metabolites disclosed herein can also be evident through an examination of the global plasma metabolome of a test subject as compared to the metabolic profile in controls. According to this approach, the overall effect of variation in metabolic processes can be seen due to the additive effect of differentiation of multiple individual metabolites across the entire metabolic processes as can be demonstrated by PCA or other comparable statistical analytical methods (FIG. 5 and FIG. 6).

Testing methods used to determine the metabolite concentration level in a sample can include those as generally known to one of skill in the art. For example, according to one embodiment, a blood sample can be obtained from a test subject and plasma can be isolated from the blood sample. The isolated blood plasma can then be examined to determine the levels of a selected panel of metabolic biomarkers in the subject and comparison of these levels with control plasma levels. When considering a plasma sample derived from a blood sample, the blood plasma will account for from about 50% to about 65% of the volume of the blood sample. As such, a plasma sample of 1 mL or less in some embodiments can be sufficient to carry out a testing protocol.

Any method of detecting the presence and concentration of the selected biomarker metabolites in the sample may be utilized, including, but not limited to, liquid chromatography-tandem mass spectroscopy (LC-MS/MS) and quadrupole-time of flight mass spectroscopy (Q-TOF MS).

According to one embodiment, a method can include treatment of an affected subject following diagnosis of ASD in the subject. Treatment can include standard behavioral modification treatments as are generally known in the art. In addition or alternatively, treatment can include modification of the metabolite level in the individual through decrease of the metabolite in the individual or through increase of the metabolite in the individual, depending upon the particular metabolite. According to one embodiment, dietary modification or drug administration may be used to mute the adverse biological effect of too much or too little of a given metabolite with or without altering the concentration of the given metabolite.

In those embodiments in which the metabolite is present in a low concentration in the affected individual as compared to a control level, the individual may be treated through supplementation of the metabolite. One or more metabolites can be supplemented to raise the levels of each of these metabolites to within normal physiological ranges for the purpose of restoring normal metabolic levels and improving ASD symptoms in the individual. Similarly, metabolites that exist at increased levels in the affected individual can be targets for metabolite decreasing treatment. For instance, activation or inhibition of key enzymes in the metabolite pathway as indicated on Table 1 previously provided, and/or utilization of an antibody or an antagonist specific for the metabolite or a key enzyme in the metabolite pathway can be used to lower levels of targeted metabolites to within normal physiological levels and improve behavioral performance of the individual.

Supplementation of a metabolite can be provided through oral administration, though any other route of supplement administration is likewise encompassed herein. For instance, an edible composition comprising the metabolite(s) for supplementation can be a dietary supplement packaged as a beverage, solid food, or semi-solid food. In some embodiments, the composition is formulated as a tablet, capsule, or gel capsule. In some embodiments, the composition can include one or more of a sweetener, a bulking agent, a stabilizer, an acidulant, and a preservative in conjunction with the metabolite(s).

In those embodiments in which the metabolite is the product of gut microbiota, adjusting the composition of gut microbiota in the subject can be utilized to vary the metabolite level in the individual. For example, the level of Clostridia bacteria, Bacterioidia bacteria, Ruminococcaceae bacteria, Erysipelotrichaceae bacteria, and/or Alcaligenaceae bacteria in the individual can be increased (for instance through the utilization of probiotic supplementation) or decreased (for instance through the utilization of an antibiotic) to adjust the composition of gut microbiota in the subject and thereby to alter the level of targeted metabolites in the subject. Various methods can be used to reduce the level of one or more bacteria species in the subject. For example, a reduced carbohydrate diet can be provided to the subject to reduce one or more intestinal bacterial species. Without being bound to any specific theory, it is believed that a reduced carbohydrate diet can restrict the available material necessary for bacterial fermentation to reduce intestinal bacterial species.

An antibody that specifically binds to a biomarker metabolite, an intermediate for the in vivo synthesis of the biomarker metabolite, or a substrate for the in vivo synthesis of the biomarker metabolite can be administered to the subject to adjust the level of the metabolite in an individual. For example, an antibody that specifically binds 12-HETE and/or one or more of the substrates and intermediates in the in vivo 12-HETE synthesis can be used to reduce the level of 12-HETE in the subject.

Methods for generating antibodies that specifically bind small molecules have been developed in the art. By way of example, an animal such as a guinea pig, rabbit, or rat, generally a mouse, can be immunized with a small molecule (e.g., the biomarker metabolite) conjugated to a hapten (e.g., KLH), the antibody-producing cells can be collected and fused to a stable, immortalized cell line, e.g., a myeloma cell line, to produce hybridoma cells which are then isolated and cloned. See, e.g., U.S. Pat. No. 6,156,882 to Buhring, et al., which is hereby incorporated by reference. In addition, the genes encoding the heavy and light chains of a small molecule-specific antibody can be cloned from a cell, e.g., the genes encoding a monoclonal antibody can be cloned from a hybridoma and used to produce a recombinant monoclonal antibody according to standard methodology as known to one of skill in the art.

Through improved diagnosis of ASD by use of the disclosed methods, individual can be treated earlier than previously possible. Moreover, through treatment of the individual via modification of the targeted metabolite levels, not only can ASD symptoms be modified, but it is believed that disease progression may be modified, leading to lifestyle improvements in affected individuals.

The present disclosure may be better understood with reference to the Examples, below.

### EXAMPLES

The examples present the results of analyses of the metabolic profile as present in plasma of individuals with ASD and normal age-matched controls.

The individuals with ASD were diagnosed following evaluation with the Autism Diagnostic Interview-Revised (ADI-R) and/or the Autism Diagnostic Observation Schedule (ADOS) and/or other Autism Diagnostic Instruments and according to the DSM IV-TR or DSM-5 criteria. Genetic tests excluded major chromosomal abnormalities, Fragile X syndrome, Rett syndrome, and abnormalities in plasma amino acid levels in the ASD subjects.

### Example 1

Plasma samples from 100 males with ASD, ages 2 to 35 years, and 32 typically developing age-matched males were included in this study. The plasma samples were stored at −20° C. until thawed for analysis. The range of freeze-thaws in the interval between collection and analysis was 1-4. The interval between collection and analysis ranged from 26 to 687 days. A principal component analysis (PCA) and a linear discriminant analysis (LDA) were carried out for the ASD and TD groups and results were compared. FIG. 5 presents the PCA results at A (top panel) and the LDA results at B (bottom panel). The samples were grouped by status as well as age, as shown. The overlap between the 2 groups is apparent primarily in the older individuals.

### Example 2

Plasma samples from 53 males with ASD ages 2-10 years and 16 typically developing age-matched males were included in this study. These samples were a sub sample of those in Example 1. The plasma samples were stored at −20° C. until thawed for analysis. The range of freeze-thaws in the interval between collection and analysis was 1-4. The interval between collection and analysis ranged from 26 to 582 days. PCA (FIG. 6 at A, top panel) and LDA testing (FIG. 6 at B, lower panel) of this cohort showed significant separation between individuals with ASD and those who were developing typically.

Twenty five metabolites which strongly discriminate children with ASD from typically developing children were determined from these data and are shown in the heat map reproduced in FIG. 4.

### Example 3

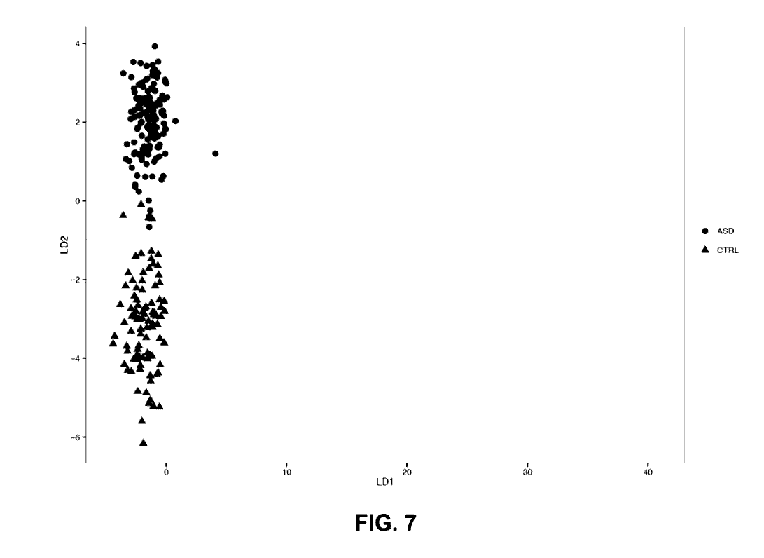

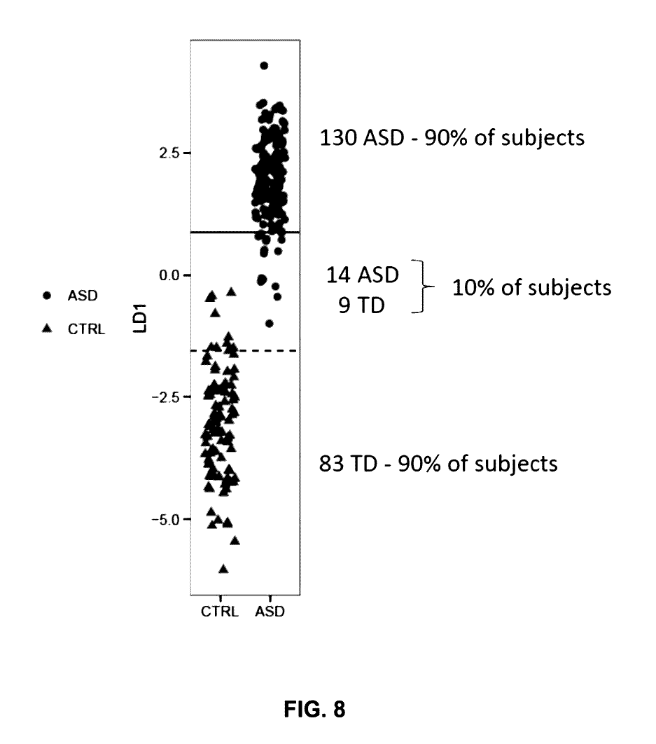

136 plasma samples from children with ASD ages 2-10 years and 92 samples from typically developing children age 2-10 years were included in this study. The sample includes those described in Example 2 plus 83 samples from children with ASD and 76 samples from children who were developing typically. The plasma samples were stored at −20° C. The number of freeze-thaws in the interval between collection and analysis was 1-4. The interval between collection and analysis ranged from 26 to 950 days. Linear discriminant analysis of global metabolic testing (see FIG. 7 and FIG. 8) showed complete separation of children with ASD from children who were developing typically.

FIG. 8 provides a partition of the linear discriminant analysis similar to results in FIG. 7 demonstrating values of typically developing children above and below the 90th centile and values of children diagnosed with ASD above and below the 10th centile.

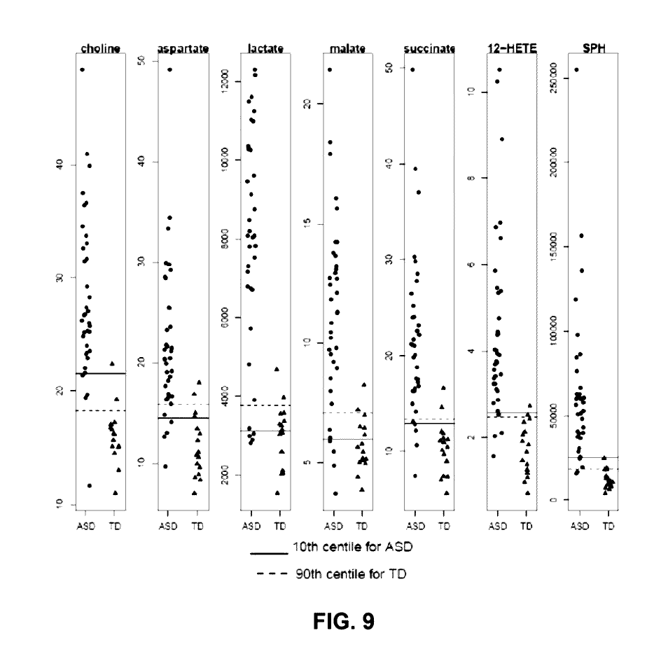

A partition of quantitative plasma concentration levels was carried out for seven different metabolites from 36 children diagnosed with ASD and 16 typically developing children. Metabolites partitioned included choline, aspartate, lactate, malate, succinate, 12-HETE, and SPH. FIG. 9 presents the results of the quantitative analysis as determined by mass spectrometry. Values above and below 10th centile for ASD-diagnosed children () are indicated by the solid line and values above and below 90th centile for typically developing children (▴) are indicated by the dashed line. Samples whose values fall above the solid line are considered to have a 90% or greater likelihood of having or developing autism. Those samples whose values fall below the dashed line are considered to represent children with a 90% or greater likelihood of developing typically.

### Example 4

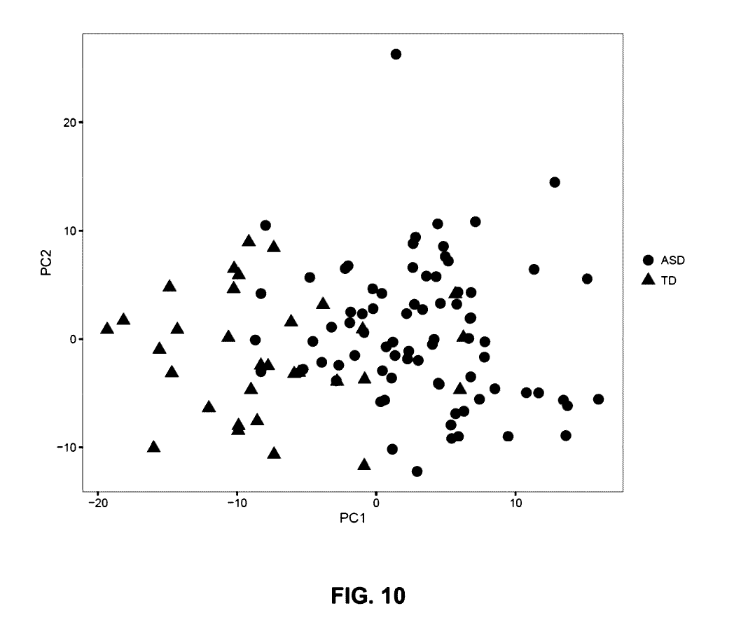

Plasma samples from 78 children, including both males and females, with ASD ages 2-5 years and 32 typically developing gender and age-matched children were included in this study. The plasma samples were stored at −20° C. until thawed for analysis. The range of freeze-thaws in the interval between collection and analysis was 1-4. Principal component analysis of global metabolomic testing of this cohort showed significant separation between individuals with ASD (circles) and those who were developing typically (triangles) (See FIG. 10). FIG. 10 includes data on 25 ASD males and 8 TD males included in data presented in FIG. 5A or FIG. 5B and data on an additional 35 ASD males ages 2-5 yrs, 18 ASD females ages 2-5 yrs, 14 TD males ages 2-5 yrs, and 10 TD females ages 2-5 years.

These and other modifications and variations to the present disclosure may be practiced by those of ordinary skill in the art, without departing from the spirit and scope of the present disclosure. In addition, it should be understood the aspects of the various embodiments may be interchanged, either in whole or in part. Furthermore, those of ordinary skill in the art will appreciate that the foregoing description is by way of example only, and is not intended to limit the disclosure.

What is claimed is:

1. A method for detection of autism spectrum disorder in a subject comprising:
obtaining a test sample from a subject, the subject being about 10 years of age or less; and
determining the concentration levels of a plurality of biomarker metabolites in the test sample, the plurality of biomarker metabolites including one of 12-HETE and 15-HETE and including one of sphingosine and choline;
wherein upon determination that the concentration level of two or more of the biomarker metabolites in the sample is about 30% or more different from a control level of the same biomarker metabolites, the subject is monitored or treated for autism spectrum disorder.

2. The method of claim 1, wherein the subject is about 5 years of age or less.

3. The method of claim 1, wherein the subject is from about 2 years of age to about 5 years of age.

4. The method of claim 1, wherein the plurality of biomarker metabolites further includes one or more of aspartate, lactate, glucose, succinate, malate, and 5-oxoproline.

5. The method of claim 4, wherein the plurality of biomarker metabolites further includes one or more of 4-hydroxyphenylpyruvate, malate, oleoylcarnitine, linoleoylcarnitine, isoleucylglycine, and valylglycine.

6. The method of claim 5, wherein the plurality of biomarker metabolites further includes one or more of 1-palmitoylglycerophosphate, lactate, fumarate, 1-arachidonoylglyercophosphate, gamma-glutamylglutamate, glucose, and uracil.

7. The method of claim 6, wherein the plurality of biomarker metabolites further includes one or more of glutamate, 2-hydroxyglutarate, xanthine, myo-inositol, and sphinganine,

8. The method of claim 7, wherein the plurality of biomarker metabolites further includes one or more of 1-oleoylplasmenylethanolamine, 4-guanidinobutanoate, S-adenosylhomocysteine, glycerate, 1-palmitoylplasmenylethanolamine, and N1-methyladenosine.

9. The method of claim 8, wherein the plurality of biomarker metabolites further includes one or more of mannose, 1-palmitoylglycerophosphate, 1-palmitoylglycerophosphocholine, 1-stearoylglycerophosphoserine, 13-NODE+9-NODE, arachidate, eicosenoate, linoleoylcarnitine, oleoylcarnitine, sphingosine 1-phosphate, stearidonate, taurine, phenylpyruvate, 5,6-dihydrouracil, orotate, gamma-glutamyllysine, valylglycine, isoleucylglycine, nicotinamide, bilirubin, and oxalate.

10. The method of claim 1, wherein the test sample comprises blood plasma.

11. The method of claim 1, further comprising treating the subject for autism spectrum disorder.

12. The method of claim 1, wherein the treatment comprises modifying the concentration of one or more of the biomarker metabolites in the subject.

13. A method for detection of autism spectrum disorder in a subject comprising:
obtaining a test sample from a subject, the subject being about 10 years of age or less; and
determining the concentration levels of a plurality of biomarker metabolites in the test sample, the plurality of biomarker metabolites including one of 12-HETE and 15-HETE and including one of sphingosine and choline;
wherein upon determination that a test value difference for two of the biomarker metabolites is about 30% or greater, the subject is monitored or treated for autism spectrum disorder, the test value being:
([M₁]/[M₂])ₜₑₛₜ/([M₁]/[M₂])_{control}
wherein
[(M₁)] is the concentration of a first biomarker metabolite; and
[(M₂)] is the concentration of a second biomarker metabolite.

14. The method of claim 13, wherein the subject is about 5 years of age or less.

15. The method of claim 13, wherein the subject is from about 2 years of age to about 5 years of age.

16. The method of claim 13, wherein the plurality of biomarker metabolites further includes one or more of aspartate, lactate, glucose, succinate, malate, and 5-oxoproline.

17. The method of claim 16, wherein the plurality of biomarker metabolites further includes one or more of 4-hydroxyphenylpyruvate, malate, oleoylcarnitine, linoleoylcarnitine, isoleucylglycine, and valylglycine.

18. The method of claim 17, wherein the plurality of biomarker metabolites further includes one or more of 1-palmitoylglycerophosphate, lactate, fumarate, 1-arachidonoylglyercophosphate, gamma-glutamylglutamate, glucose, and uracil.

19. The method of claim 18, wherein the plurality of biomarker metabolites further includes one or more of glutamate, 2-hydroxyglutarate, xanthine, myo-inositol, and sphinganine,

20. The method of claim 19, wherein the plurality of biomarker metabolites further includes one or more of 1-oleoylplasmenylethanolamine, 4-guanidinobutanoate, S-adenosylhomocysteine, glycerate, 1-palmitoylplasmenylethanolamine, and N1-methyladenosine.

21. The method of claim 20, wherein the plurality of biomarker metabolites further includes one or more of mannose, 1-palmitoylglycerophosphate, 1-palmitoylglycerophosphocholine, 1-stearoylglycerophosphoserine, 13-NODE+9-NODE, arachidate, eicosenoate, linoleoylcarnitine, oleoylcarnitine, sphingosine 1-phosphate, stearidonate, taurine, phenylpyruvate, 5,6-dihydrouracil, orotate, gamma-glutamyllysine, valylglycine, isoleucylglycine, nicotinamide, bilirubin, and oxalate.

22. The method of claim 13, wherein the test sample comprises blood plasma.

23. The method of claim 13, further comprising treating the subject for autism spectrum disorder.

24. The method of claim 13, wherein the treatment comprises modifying the concentration of one or more of the biomarker metabolites in the subject.

29. A method for diagnosis of autism spectrum disorder in a subject comprising:
obtaining a test sample from a subject, the subject being about 10 years of age or less; and
determining a global metabolic level of the subject from the test sample;
wherein upon determination that the global plasma metabolome of the test sample is about 30% or more different from a control level of a control global plasma metabolome, the subject is monitored or treated for autism spectrum disorder.

30. The method of claim 29, wherein the determination is carried out according to a principal component analysis.

31. The method of claim 29, wherein the subject is about 5 years of age or less.

32. The method of claim 29, wherein the subject is from about 2 years of age to about 5 years of age.

33. The method of claim 29, wherein the test sample comprises blood plasma.

34. The method of claim 29, further comprising treating the subject for autism spectrum disorder.

35. The method of claim 29, wherein the treatment comprises modifying the concentration of one or more biomarker metabolites in the subject.
